(19) United States
(12) Patent Application Publication
(10) Pub. No.: US 2024/0342198 A1
(43) Pub. Date: Oct. 17, 2024

(54) TREATMENT OF IRRITABILITY IN SUBJECTS WITH AUTISM SPECTRUM DISORDERS WITH MODERATE TO SEVERE ANXIETY AND/OR SOCIAL AVOIDANCE

(71) Applicant: ZYNERBA PHARMACEUTICALS, INC., Devon, PA (US)

(72) Inventors: Joseph Palumbo, Saint Davids, PA (US); Stephen V. O'Quinn, Wake Forest, NC (US)

(73) Assignee: ZYNERBA PHARMACEUTICALS, INC., Devon, PA (US)

(21) Appl. No.: 18/642,059

(22) Filed: Apr. 22, 2024

Related U.S. Application Data
(63) Continuation of application No. PCT/US2022/078449, filed on Oct. 20, 2022.
(60) Provisional application No. 63/271,015, filed on Oct. 22, 2021.

(51) Int. Cl. A61K 31/00; A61P 25/00

(52) U.S. Cl. CPC A61K 31/658; A61P 25/00

(57) ABSTRACT

The present technology relates to a method of treating one or more behavioral symptoms of autism spectrum disorder (ASD) in a subject by administering an effective amount of cannabidiol (CBD). Specifically, subjects having moderate to severe ASD and relatively high social avoidance and/or anxiety are more likely to show a reduction in irritability when treated with CBD.

### RELATED APPLICATIONS

This application is a continuation of International Application No. PCT/US2022/078449, filed Oct. 20, 2022 which claims priority to U.S. Provisional Patent Application 63/271,015, filed on Oct. 22, 2021, the contents of each are hereby incorporated by reference in their entirety.

### FIELD OF THE TECHNOLOGY

The present disclosure relates to methods of treating irritability in a subject diagnosed with autism spectrum disorder (ASD) by administering an effective amount of cannabidiol (CBD) to the subject wherein the irritability symptoms of ASD are treated in the subject.

### BACKGROUND

Autism spectrum disorders (ASD) are generally characterized by a number of neurodevelopmental impairments such as difficulties in communication and socialization (e.g., social avoidance/withdrawal), and rigid, repetitive behaviors. “Problem behaviors” are also very common in ASD and can be more severe in ASD compared to typical development than the neurodevelopmental impairments. Problem behaviors include self-injury, running away, aggression, property damage, inappropriate behavior/language and irritability. In addition, many children diagnosed with ASD meet the criteria for anxiety disorder. For children diagnosed with ASD, the management of problem behaviors can be challenging for the adult caregivers. (See O'Nions et al. “How do Parents Manage Irritability, Challenging Behaviour, Non-Compliance, and Anxiety in Children with Autism Spectrum Disorders? A meta-Synthesis” J. of Autism and Developmental Disorders (2018) 48:1272-1286).

Caregivers of children diagnosed with ASD typically deal with problem behaviors by trying to adapt the environment of the child to avoid situations that could give rise to problem behaviors. Another way to handle problem behavior is to anticipate and prepare strategies for handling problem behaviors. These concessionary strategies, however, do little to reduce the incidence of problem behaviors. There is, therefore, a need for treatments that are safe and effective for reducing problem behaviors in children diagnosed with ASD.

### SUMMARY

Handling behavior problems in subjects diagnosed with moderate to severe ASD can be challenging for caregivers. One such problem behavior is high irritability. High irritability can present as anger, frustration, distress, and meltdowns. Frequent episodes of high irritability can lead to considerable challenges for caregivers.

Unexpectedly, it has been found that response to CBD treatment of irritability in patients diagnosed with moderate to severe ASD is enhanced in subjects that also exhibit high social avoidance scores and/or high anxiety scores, relative to the general population of ASD subjects and Fragile X Syndrome (FSX) subjects who have symptoms of ASD. Specifically, about twice as many subjects that exhibit high social avoidance scores, high anxiety scores, or both, relative to the general population of ASD subjects, showed an improvement in irritability compared to subjects that had lesser avoidance and anxiety scores. These results show that the use of CBD for the treatment of irritability in ASD subjects is particularly effective in the subsets of these subjects that also exhibit high anxiety and/or high social avoidance.

In an embodiment, irritability in a subject diagnosed with autism spectrum disorder (ASD) may be treated by administering an effective amount of cannabidiol (CBD) to the subject. The administration of CBD improves the irritability of the subject, based on an ABC-C Irritability score. In an embodiment, the subject, before treatment, has an ABC-C irritability score greater than or equal to 12.

In another embodiment, the subject diagnosed with ASD, along with relatively high irritability, may exhibit relatively high social avoidance and/or relatively high anxiety. A subject diagnosed with moderate to severe ASD, in some embodiments, has an Autism Diagnostic Observation Schedule®, 2nd Edition (ADOS-2) comparison score of greater than or equal to 3. In some embodiments, a subject having relatively high social avoidance has an ABC-C social avoidance score of greater than 5. In some embodiments, a subject having relatively high anxiety has a Parent Rated Anxiety Scale for ASD (PRAS-ASD) score of greater than 25.

In an embodiment, the CBD is synthetic CBD. Alternatively, the CBD may be botanically derived CBD that is unpurified or purified. The CBD may be administered orally or transdermally. In an embodiment, botanically obtained CBD does not contain THC. The effective amount of CBD may be 250 mg/day; 500 mg/day; or 750 mg/day. In some embodiments, an effective amount of CBD may be administered once/day or twice/day.

In some embodiments, the subject is diagnosed with Fragile X Syndrome (FXS), comorbid with ASD.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 is a plot of PRAS Total Score (Baseline) vs. ABC-C Social Avoidance subscale (Baseline).

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION

As used herein, the term “treating” or “treatment” refers to mitigating, improving, relieving, or alleviating at least one symptom (such as a behavioral symptom) of a condition, disease or disorder in a subject, such as a human, or the improvement of an ascertainable measurement associated with a condition, disease or disorder.

As used herein, the term “clinical efficacy” refers to the ability to produce a desired effect in humans as shown through human clinical studies or trials.

As used herein, the term “cannabidiol” or “CBD” refers to cannabidiol (2-[3-methyl-6-(1-methylethenyl)-2-cyclohexen-1-yl]-5-pentyl-1,3-benzenediol); cannabidiol prodrugs; and pharmaceutically acceptable salts, solvates, metabolites, and metabolic precursors thereof. CBD may be obtained and purified from plant material or synthesized. The synthesis of CBD is described, for example, in Petilka et al., Helv. Chim. Acta, 52:1102 (1969) and in Mechoulam et al., J. Am. Chem. Soc., 87:3273 (1965), which are both hereby incorporated by reference. In preferred embodiments, optically active (−)-CBD is used in the therapeutic treatments described herein.

As used herein, the term “transdermally administering” refers to contacting the patient's or subject's skin with a composition comprising an active agent under conditions effective for the active agent to penetrate the skin.

Autism Spectrum Disorder (ASD) is a developmental disorder that affects communication and behavior in approximately one million pediatric and adolescent patients between the ages of five and 17 in the U.S. ASD refers to a range of conditions characterized by anxiety, repetitive patterns of behavior, impairments in social communication including verbal and non-verbal communication, and deficits in developing and maintaining relationships. Although autism can be diagnosed at any age, it is said to be a “developmental disorder” because symptoms generally appear in the first two years of life. Research suggests that genes can act together with influences from the environment to affect development in ways that lead to ASD. Newer studies suggest that ASD is linked to disruption in the endocannabinoid system.

The severity of symptoms of ASD in a subject is typically performed by looking at a person's behavior and development. A number of behavioral tests have been developed to help clinicians measure the severity of behavioral symptoms of ASD. Exemplary tests to help clinicians determine the severity of symptoms of ASD include, but are not limited to, the following tests: Anxiety, Depression, and Mood Scale (ADAMS), Aberrant Behavior Checklist (ABC); Aberrant Behavior Checklist-Community (ABC-C); Diagnostic and Statistical Manual of Mental Disorders, Fifth Edition (DSM-5), Diagnostic and Statistical Manual of Mental Disorders, Fifth Edition, Text Revisions (DSM-5-TR); and Autism Diagnosis Observation Schedule-Second Edition (ADOS-2).

The Anxiety, Depression, and Mood Scale (ADAMS) is a behavioral test that is used by clinicians, doctors, and researchers to assess the level of anxiety, depression and mood in patients with intellectual disabilities, including ASD. The ADAMS test consists of questions grouped into five subscales, including (i) general anxiety, (ii) social avoidance, (iii) compulsive behavior, (iv) manic/hyperactive behavior, and (v) depressed mood. Each question is answered by a clinician/doctor on a four-point scale ranging from 0 (“not a problem”) to 3 (“severe problem”). In addition to subscale scores, the ADAMS yields a total score.

Another test that can be used by clinicians to measure the severity of symptoms of ASD is the Aberrant Behavior Checklist-Community test (ABC-C). The original Aberrant Behavior Checklist (ABC) was designed to assess behavioral concerns of adults within institutional settings. The original ABC was later adapted to address patients who are not institutionalized and specifically to address subjects diagnosed with ASD. The Aberrant Behavior Checklist-Community (ABC-C) is used by clinicians, doctors, and researchers to access certain behaviors in non-institutionalized patients with ASD. The original ABC-C test has five subscales which include:

(i) irritability, (ii) hyperactivity, (iii) social withdrawal, (iv) stereotypical behavior, and (v) inappropriate speech. Similar to ADAMS, the ABC-C scale is a four-point Likert-type scale ranging from 0 (not a problem) to 3 (problem is severe). The ABC-C irritability subscale was used as the basis for approval for the two atypical antipsychotics indicated for ASD. A modified score of the ABC-C was created to better assess behaviors in subjects diagnosed with FXS (ABC-CFXS) which uses the same questions, but breaks the scoring into six subscales with the addition of a subscale for social avoidance.

The present disclosure also relates to a method of treating irritability symptoms of Autism Spectrum Disorder (ASD) in a subject by administering an effective amount of cannabidiol (CBD) to the subject wherein irritability symptoms of ASD are treated in the subject. The method includes transdermally administering an effective amount of cannabidiol (CBD) to the subject.

In an embodiment, the method can be used to treat irritability in a subject having moderate to severe ASD. A subject having moderate to severe ASD has an Autism Diagnostic Observation Schedule®, 2nd Edition (ADOS-2) comparison score of greater than or equal to 3.

Generally, a subject experiencing “high irritability” will experience irritability that is greater than a mean irritability of the relevant general population. Irritability can be determined using a scale such as the ABC-C Irritability subscale. A subject that is in need of treatment for high irritability has a high ABC-C Irritability score. As used herein, the term “high ABC-C Irritability score” refers to an ABC-C Irritability score of greater than 12, greater than 13, greater than 14, greater than 15, greater than 16, greater than 17, greater than 18, greater than 19 or greater than 20.

Generally, a subject experiencing “high social avoidance” will experience social avoidance that is greater than a mean social avoidance of the relevant general population. Social Avoidance can be determined using a scale such as the ABC-CFXS Social Avoidance subscale. A subject that has “high” social avoidance has an ABC-CFXS Social Avoidance score of greater than 5, greater than 6, greater than 7, greater than 8, or greater than 9.

Generally, a subject experiencing “high anxiety” will experience anxiety that is greater than a mean anxiety of the relevant general population. Anxiety can be determined using a scale such as the Parent Rated Anxiety Scale for ASD (PRAS-ASD). A subject that has “high” anxiety has a PRAS-ASD score of greater than 25, greater than 30, greater than 35, greater than 37, greater than 40, or greater than 45.

It should be understood that scores used to determine the high irritability, high anxiety, and high social avoidance are used to show an association with statistical data and clinical evaluation by physicians. A stated score associated with higher than usual behavior (e.g., high irritability, high social avoidance, and high anxiety) is a population estimate from studies that show that the clinical evaluation of such behaviors is associated with these scores, however, it is not necessarily an absolute threshold. Therefore, it should be understood that a physician does not necessarily rely on specific scores related to irritability, social avoidance, and anxiety to determine that a patient would benefit from pharmaceutical treatment. Rather use of a clinical evaluation, with or without determining specific scores associated with the behavior, may lead a physician to conclude that a subject is experiencing high irritability, high social avoidance, and high anxiety.

Therapeutic medicines have been developed that utilize innovative transdermal technologies to allow for sustained and controlled delivery of therapeutic levels of CBD. Transdermal cannabidiol delivery systems are taught in U.S. Pat. Nos. 8,449,908 and 8,435,556, both of which are incorporated herein by reference.

Transdermal delivery of cannabinoids (e.g., CBD) has benefits over oral dosing because it allows the drug to be absorbed through the skin directly into the bloodstream. This avoids first-pass liver metabolism, potentially enabling lower dosage levels of active pharmaceutical ingredients with a higher bioavailability and improved safety profile. Transdermal delivery also avoids the gastrointestinal tract, lessening the opportunity for GI related adverse events and the potential degradation of CBD by gastric acid into THC, which can be associated with unwanted psychoactive effects. Moreover, transdermal delivery of CBD reduces the intensity and frequency of somnolence as an adverse event, which are typically present in oral dosing of CBD. Transdermal delivery of CBD can avoid liver function adverse events, which are typically present in oral dosing of CBD. In some embodiments, transdermally administering an effective amount of CBD reduces an intensity of at least one adverse event by about 15% to about 95% relative to orally administering CBD.

The effective amount of CBD can be between about 50 mg to about 1000 mg daily. In some embodiments, the effective amount of CBD is initiated at about 50 mg daily and titrated up to about 750 mg daily. The effective amount of CBD can be initiated at about 50 mg daily and titrated up to about 250 mg daily, 500 mg daily, 750 mg daily, or 1000 mg daily. In some embodiments, the effective amount of CBD is initiated at 250 mg daily. The effective amount of CBD can be initiated at 500 mg daily. The effective amount of CBD can be initiated at 750 mg daily. The effective amount of CBD can be initiated at 1000 mg daily. In some embodiments, a daily dose of about 250 mg is administered to patients that weigh less than, or equal to, 35 kg. In some embodiments, a daily dose of about 500 mg is administered to patients that weigh more than 30 kg and less than, or equal to, 50 kg. In some embodiments, a daily dose of about 750 mg is administered to patients that weigh more than 50 kg. CBD can be administered in a single daily dose or in two daily doses. In some embodiments, the effective amount of CBD can be 390 mg in divided daily doses.

The CBD can be in a gel form and can be pharmaceutically-produced as a clear, permeation-enhanced gel that is designed to provide controlled drug delivery transdermally with once- or twice-daily dosing. The CBD gel can between 1% (wt/wt) CBD to 7.5% (wt/wt) CBD. The CBD gel can have, for example, 4.2% (wt/wt) CBD or 7.5% (wt/wt) CBD). The CBD gel can be applied topically by the patient or caregiver to the patient's upper arm and shoulder, back, thigh, or any combination thereof.

The CBD gel can include diluents and carriers as well as other conventional excipients, such as wetting agents, preservatives, and suspending and dispersing agents.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

In some embodiments, the transdermal preparation can be a cream, a salve or an ointment. The CBD can be delivered by a bandage, pad or patch. The CBD can be administered transdermally on the subject's upper arm and shoulder. In some embodiments, the CBD is administered transdermally on the subject's thigh or back. The CBD can be synthetic CBD. The CBD can be purified CBD. The CBD can be botanically derived.

In some embodiments, the CBD is administered in a pharmaceutically acceptable preparation that does not contain THC. In some embodiments, the CBD is administered without THC or any other extracts of cannabis. In some embodiments, the CBD is synthetic CBD. In some embodiments it an extract. In some embodiments, it is purified.

Alleviating irritability in a subject diagnosed with Autism Spectrum Disorder (ASD) can include an improvement in an ABC-C irritability score. Irritability can be measured using the ABC-C irritability subset score. High irritability in a subject may be indicated if the ABC-C irritability score is equal to or greater than 18. In an embodiment, an improvement in the ABC-C irritability score is indicated when the ABC-C irritability score of a subject is less than 18, or less than 17, or less than 16, or less than 15, or less than 14, or less then 13, or less then 12, or less than 11, or less than 10, or less than 9, or less then 8, or less than 7, or less than 6, or less than 5, or less than 4, or less than 3, or less than 2, or less than 1, or equal to 0, after treatment of the subject with CBD. In an embodiment, an improvement in the ABC-C irritability score is indicated when the ABC-C irritability score in a subject, after treatment with CBD, is reduced by at least 3, or reduced by at least 4, or reduced by at least 5, or reduced by at least 6, or reduced by at least 7, or reduced by at least 8, or reduced by at least 9, or reduced by at least 10, or reduced by at least 11, or reduced by at least 12, or reduced by at least 13, or reduced by at least 14, or reduced by at least 15, or reduced by at least 16, or reduced by at least 17, or reduced by at least 18. In an embodiment, an improvement in the ABC-C irritability score is indicated when the ABC-C irritability score in a subject, after treatment with CBD, is reduced by at least 5%, or reduced by at least 10%, or reduced by at least 15%, or reduced by at least 20%, or reduced by at least 25%, or reduced by at least 30%, or reduced by at least 35%, or reduced by at least 40%, or reduced by at least 45%, or reduced by at least 50%, or reduced by at least 55%, or reduced by at least 60%.

Alleviating irritability in a subject diagnosed with moderate to severe ASD can also include an improvement in a total score of an Anxiety, Depression and Mood Scale (ADAMS). In some embodiments, alleviating one or more behavioral symptoms of ASD can include improvement in one or more subscales of ADAMS.

In some embodiments, the subject is also being administered one or more additional medications. The one or more additional medications, in some embodiments, are selected from the group consisting of an anti-depressant, an anxiolytic, an alpha-2-adrenergic agonist, a psychostimulant, an antipsychotic medication, and combinations thereof.

In some embodiments, the one or more additional medications include an antipsychotic medication. Examples of antipsychotic medications typically administered to subjects diagnosed with ASD include, but are not limited to, risperidone, aripiprazole, haloperidol, olanzapine, ziprasidone, and quetiapine fumarate in some embodiments.

In some embodiments, the one or more additional medications include an alpha-2-adrenergic agonist. Examples of alpha-2-adrenergic agonists typically administered to subjects diagnosed with ASD include, but are not limited to, clonidine and guanfacine.

In some embodiments, the one or more additional medications include an anti-depressant. For example, a selective serotonin reuptake inhibitor (SSRI) anti-depressant may be also be administered to a subject as an additional medication. Examples of SSRIs that are used in subjects with ASD include, but are not limited to, fluoxetine, citalopram, and escitalopram.

In some embodiments, the one or more psychotropic medications include a psychostimulant medication. Examples of psychostimulant medications typically administered to subjects diagnosed with ASD include, but are not limited to, methylphenidate HCl, atomoxetine HCl, dexamfetamine, and lisdexamfetamine mesilate.

Unexpectedly, it has been found that treatment of irritability in a subject diagnosed with ASD is enhanced if the patient has a high social avoidance score and/or a high anxiety score. Typically, the severity of symptoms in patients with ASD are determined using the ABC-C test. As discussed before, the ABC-C test has five subscales which include (i) irritability, (ii) hyperactivity, (iii) social withdrawal, (iv) stereotypical behavior, and (v) inappropriate speech. The ABC-CFXS test was created to better assess behaviors of subjects diagnosed with FXS. The ABC-CFXS test uses the same questions as the ABC-C test, but breaks the scoring into six subscales with the addition of a subscale for social avoidance. When the ABC-CFXS test is applied to subjects with ASD (but not a diagnosis of FXS), this unique application of the ABC-CFXS test provides a novel separation of subjects based on criteria previously not used to study subjects with ASD, (i.e., social avoidance). When applying the ABC-CFXS test to ASD subjects, it was found that a subject having an ABC-CFXS Social Avoidance score of greater than 7 showed twice as much improvement in irritability, compared to subjects having an ABC-CFXS social avoidance score of less than, or equal to 7. By modifying the existing testing standards for the severity of symptoms with patients diagnosed with ASD (ABC-C) with a standard that was developed for patients diagnosed with FXS (ABC-CFXS), a preferential and positive response to treatment with CBD was discovered for irritability in subjects having high ABC-CFXS social avoidance scores.

It was also found that subjects having a Parent Rated Anxiety Scale (PRAS) score of greater than 37 showed twice as much improvement in irritability, compared to subjects having a PRAS score of less than, or equal to 37.

In some embodiment, the subject may be diagnosed with Fragile X Syndrome (FXS) comorbid with moderate to severe ASD. Similar to subjects that have a diagnosis of ASD, patients diagnosed with FXS and ASD show improved irritability when treated with CBD, particularly transdermal CBD. It was also found that FXS subjects having high social avoidance scores and/or high anxiety scores also show the same enhanced reduction in irritability as seen with ASD patients without FXS.

### EXAMPLES

### Example 1: Treatment of ASD—BRIGHT Study

An exploratory open-label safety, tolerability and efficacy study of Zygel™ ZYNO02 transdermal gel to 37 children and adolescents with autism spectrum disorder was conducted. The patient population (ages 4 through 17 years old) were predominantly moderate-to-severe ASD patients. ASD was confirmed by Diagnostic and Statistical Manual of Mental Disorders, 5th edition (DSM-5) diagnostic criteria to assess the safety and efficacy of ZYNO02 in treating ASD-related behaviors as measured by a variety of efficacy assessments. These included the Aberrant Behavior Checklist-Community (ABC-C); the Autism Diagnostic Observation Schedule®, 2nd Edition (ADOS-2); and the Parent Rated Anxiety Scale-Autism Spectrum Disorder (PRAS-ASD). ZYNO02 was administered to patients with moderate-to-severe symptoms of ASD as add-on therapy to their standard of care.

### Patient Demographics

The majority of the patients were male (92%) with a mean age of 9.2 years. Patients weighed between 15 and 108 kilograms (mean=41.6; median=30.2). The mean time to diagnosis in this population was 5.4 years. The majority of patients had moderate or severe ASD at baseline as measured by the ADOS®-2 comparison score (94%) and Diagnostic and Statistical Manual of Mental Disorders, 5th edition, severity levels (92%). The mean ABC-C Irritability score was 30.3, and 9 patients (24.3%) had PRAS-ASD scores indicative of possible clinical anxiety, further highlighting the severity of symptoms in the enrolled patient population.

The majority (92%) of patients entered the trial with the use of at least one underlying medication. 65% of patients were on at least one psychotropic medication, e.g., anti-depressants, anxiolytics, and antipsychotics. 14 of the 37 subjects were on antipsychotics, 11 on risperidone, 1 on haloperidol, 1 on olanzapine, and 1 on quetiapine fumarate. 16 were on psychostimulant agents used for ADHD and nootropics, including clonidine (6), guanfacine (5), methylphenidate HCl (7), atomoxetine HCl (2), dexamfetamine (1), and lisdexamfetamine mesilate (2).

### Protocol

Subjects were administered a 250 or 500 mg total daily dose, administered twice daily, of CBD in the form of ZYN002 CBD transdermal gel for 14 weeks. After completing dosing in the 14-week period, participants who qualified were given the option to enroll in a six-month extension trial.

The trial evaluated multiple efficacy assessments, including the ABC-C, PRAS-ASD, Autism Parenting Stress Index, Autism Impact Measure (AIM), and Clinical Global Impression-Severity (CGI-S) and Improvement (CGI-I). The ABC-C irritability subscale was used as the basis for approval for the two atypical antipsychotics indicated for ASD (risperidone and aripiprazole).

### Results

All five subscales of the ABC-C as well as the Parent Rated Anxiety Scale-Autism Spectrum Disorder (PRAS-ASD) showed both statistically significant and clinically meaningful improvements at 14 weeks of treatment versus baseline.

Table 1 summarizes the 14-week improvement from each of the subscales of the ABC-C. All results were statistically significant; p<0.001 for all subscales.

TABLE 1
ABC-C Improvement at 14 Weeks

| | Baseline | Week 14 | Mean % |
|---|---|---|---|
| | (n = 36) | (n = 28) | improvement |
| ABC: Irritability | 30.3 | 18.2 | 39.1% |
| ABC: Inappropriate Speech | 7.4 | 5.2 | 42.5% |
| ABC: Stereotypy | 12.3 | 7.9 | 39.1% |
| ABC: Social withdrawal | 25.1 | 16.5 | 36.4% |
| ABC: Hyperactivity | 37.0 | 23.9 | 35.6% |

There was a 40% improvement in stereotypic behavior on the ABC scale, a 33% improvement in repetitive behavior on Parent Reported Anxiety Scale, and an unexpected overall improvement in children with this severity of ASD and who were also on antipsychotic medications. The results are both statistically significant and clinically meaningful.

The results of other efficacy assessments reinforce the results demonstrated in the ABC-C. For example, patients on ZYNO02 experienced a mean improvement of 46% at week 14 from a baseline score of 40.8 as measured by the PRAS-ASD (p<0.001) and 57% of patients were assessed as very much or much improved at week 14 as measured by the Clinical Global Impressions-Improvement scale (CGI-I).

ZYNO02 was well tolerated in this trial with no serious adverse events (SAE) reported. Twenty-eight patients completed the 14-week trial; this discontinuation rate is consistent with other trials in ASD. Only one patient was lost to follow up with no post-treatment efficacy evaluation. Less than half (49%) of the patients experienced any adverse event (non-related or related to study drug), all of which were mild (75%) or moderate (25%). Only 14% of patients experienced an adverse event deemed to be treatment-related, all of which were application site-related and most were mild and transient. There were no severe adverse events reported during the study. Eighteen (18) patients who completed the BRIGHT trial enrolled in the open label extension.

### Results

All five subscales of the ABC-C as well as the Parent Rated Anxiety Scale-Autism Spectrum Disorder (PRAS-ASD) showed both statistically significant and clinically meaningful improvements at 14 weeks of treatment versus baseline. Table 1 summarizes the week 6 and week 14 ABC-C results.

### Example 4: Treatment of Irritability in Subjects

In data obtained from the previously described trials, it was noted that administration of transdermal CBD had a significant improvement in the irritability score of subjects diagnosed with moderate to severe ASD. A summary of the results for the completers (N=28) from the study for the open-label safety, tolerability and efficacy study of Zygel™ ZYN002 transdermal gel (See Example 1) are presented in Table 1.

The data collected from the studies presented herein, and other similar studies were pooled together and analyzed. Table 2 presents a summary of pooled data collected from 156 subjects. The subjects had a baseline ABC-C Irritability score of greater than, or equal to, 18. The subjects had moderate to severe symptoms of ASD (ADOS-2, comparison score greater than, or equal to, 5). The data presented in Table 2 is the Week 12 change in ABC-C Irritability of all patients. In previous studies (such as presented in Example 1, Table 1) the data was enriched by removing the non-completers from the analysis and only presenting data from the completers. The current analysis in Table 2 shows that, without data enrichment, Zygel™ ZYNO02 transdermal gel provided a minimal improvement, compared to placebo, of ABC-C Irritability in patients with FXS and comorbid ASD.

TABLE 2

| Parameter Time | Placebo | ZYN002 | Total |
|---|---|---|---|
| Point Statistic | (N = 73) | (N = 83) | (N = 156) |
| N | 73 | 80 | 153 |
| Mean | −2.89 | −4.80 | −3.89 |
| SD (SE) | 7.17 (0.84) | 7.09 (0.79) | 7.17 (0.58) |
| Median | −2.0 | −5.0 | −3.0 |
| Min, Max | −23, 17 | −31, 10 | −31, 17 |

The data presented in Table 2 was further analyzed by limiting the data to subjects that had a baseline ABC-C Irritability score of greater than, or equal to, 18 and an ABC-CFXS Social Avoidance score of greater than 7. These results were compared to patents that had a baseline ABC-C Irritability score of greater than, or equal to, 18 and an ABC-CFXS Social Avoidance score of less than, or equal to 7. The results of this analysis are presented in Table 3. This data shows that subjects having an ABC-CFXS Social Avoidance score of greater than 7 had a much better response to transdermal CBD administration than subjects having an ABC-CFXS Social Avoidance score of less than, or equal to 7.

TABLE 3

| | Parameter Time | | |
|---|---|---|---|
| | Point Statistic | Placebo | ZYN002 |
| SA > 7 | N | 29 | 27 |
| | Mean | −3.34 | −7.96 |
| | SD (SE) | 7.66 (1.42) | 9.46 (1.82) |
| | Median | 0.0 | −6.0 |
| | Min, Max | −25, 4 | −31, 10 |
| SA ≤ 7 | N | 23 | 35 |
| | Mean | −5.57 | −4.74 |
| | SD (SE) | 6.8 (1.42) | 6.03 (1.02) |
| | Median | −5.0 | −4.0 |
| | Min, Max | −23, 6 | −17, 6 |

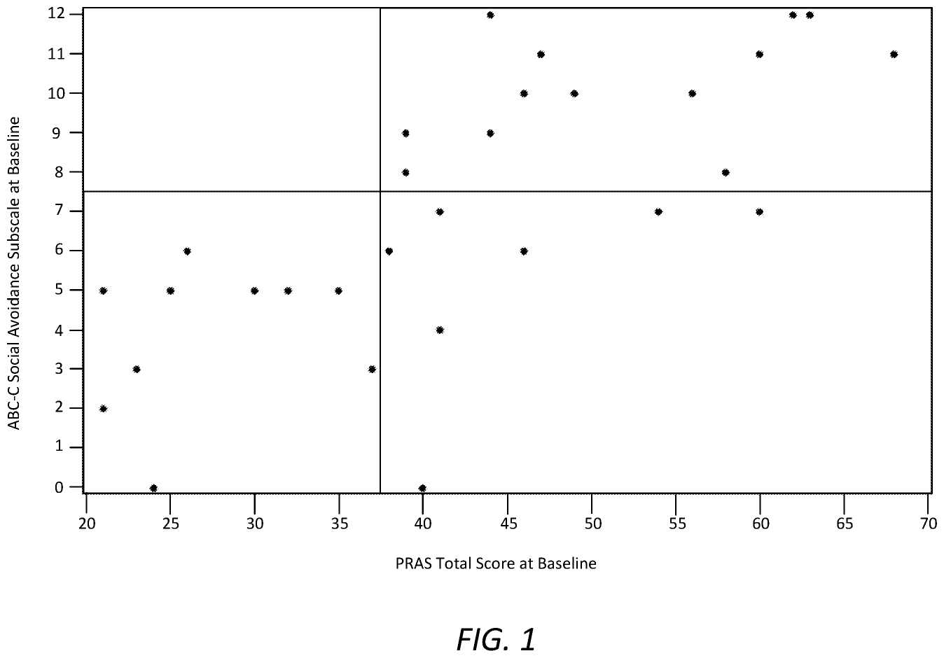

Further, data from the BRIGHT study in ASD, showed that baseline anxiety scores on the Parent Rated Anxiety Scale for ASD (PRAS-ASD) were found to be correlated with ABC-CFXS Social Avoidance. FIG. 1 shows a plot of PRAS Total Score (Baseline) vs. ABC-CFXS Social Avoidance subscale (Baseline). The data shows correlation between social avoidance and anxiety, with a correlation coefficient of 0.45526 (P value=0.005).

This led to further analysis of the Zygel™ ZYNO02 transdermal gel data to see the effect of Zygel on ABC-C Irritability score in subjects having high PRAS-ASD anxiety scores (greater than 37). This analysis, presented in Table 4, shows that subjects with baseline ABC-C Irritability score of greater than, or equal to, 18 and PRAS-ASD scores greater than 37 showed better improvement of irritability compared to subjects with a PRAS-ASD score less than, or equal to, 37. The improvement in irritability in subjects with high PRAS-ASD scores is summarized in Table 4. Note that subjects in Period 2 were responders from Period 1 (defined as ≥35% improvement in the ABC-C Irritability score from baseline to week 14 in Period 1) and as such may have even greater change than all subject in Period 1 as a result.

TABLE 14
Summary of Mean Change from Baseline (BL) to Week
14 by ABC-C Irritability by Baseline PRAS Score

| Baseline PRAS Score | N, Change (SD) |
|---|---|
| >37 | 18, −14.7 (8.3) |
| ≤37 | 10, −7.6 (9.4) |

Collectively, this data shows that subjects having moderate to severe ASD and relatively high social avoidance and/or anxiety are more likely to show a reduction in irritability when treated with CBD.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of treating irritability in a subject diagnosed with autism spectrum disorder (ASD), the method comprising:
administering an effective amount of cannabidiol (CBD) to the subject, wherein the subject has a high baseline ABC-C irritability score, and wherein the subject also has a high baseline ABC-CFXS social avoidance score and/or a high baseline Parent Rated Anxiety Scale for ASD (PRAS-ASD),
wherein irritability in the subject is treated.

2. The method according to claim 1, wherein the subject has a baseline ABC-C irritability score of greater than 12.

3. The method according to claim 1, wherein the subject has a baseline ABC-CFXS social avoidance score of greater than 5.

4. The method according to claim 1, wherein the subject has a baseline Parent Rated Anxiety Scale for ASD (PRAS-ASD) score of greater than 25.

5. The method according to claim 1, wherein the subject is diagnosed with moderate to severe ASD.

6. The method according to claim 1, wherein the subject has an Autism Diagnostic Observation Schedule, 2nd Edition (ADOS-2) comparison score of greater than or equal to 3.

7. The method according to claim 1, wherein the CBD is administered transdermally.

8. The method according to claim 1, wherein the CBD is synthetic CBD.

9. The method according to claim 1, wherein the CBD is botanically derived CBD.

10. The method according to claim 1, wherein the effective amount of CBD is a 250 mg total daily dose.

11. The method according to claim 1, wherein the effective amount of CBD is a 500 mg total daily dose.

12. The method according to claim 1, wherein the effective amount of CBD is a 750 mg total daily dose.

13. The method according to claim 1, wherein the effective amount of CBD is a 1000 mg total daily dose.

14. The method according to claim 1, wherein the effective amount is administered in two daily doses.

15. The method according to claim 1, wherein the CBD is administered in a pharmaceutically acceptable preparation that does not contain THC.

16. The method according to claim 1, wherein the subject is also diagnosed with Fragile X Syndrome (FXS).
